(19) United States
(12) Patent Application Publication
(10) Pub. No.: US 2010/0116790 A1
(43) Pub. Date: May 13, 2010

(54) Device and method for locally producing microwave plasma

(75) Inventor: Ralf Spitzl, Troisdorf (DE)

Correspondence Address: D. PETER HOCHBERG CO. L.P.A., 1940 EAST 6TH STREET, CLEVELAND, OH, 44114, US

(73) Assignee: iplas Innovative Plasma Systems GmbH, Troisdorf (DE)

(21) Appl. No.: 12/311,881

(22) PCT Filed: Oct. 11, 2007
(86) PCT No.: PCT/EP2007/008840

§ 371 Date: Jul. 20, 2009

(30) Foreign Application Priority Data
Oct. 16, 2006 (DE) 10 2006 048 816.4

(51) Int. Cl. H01J 37/32; H01L 21/3065; H05H 1/00; C23C 16/50; A61L 2/14; C23F 1/00; D06B 19/00; A61L 9/22; C23C 16/511; C23C 16/513

(52) U.S. Cl. 216/69; 156/345.37; 156/345.36; 427/575; 134/1.1; 422/22; 8/115.52; 422/4; 422/186.04

(57) ABSTRACT

A device for locally producing microwave plasma. The device comprises at least one microwave feed that is surrounded by at least one dielectric tube. At least one of the dielectric tubes, such as an outer dielectric tube, is partially surrounded by a metal jacket. A locally delimited plasma is produced by the device by shielding microwaves.

### CROSS-REFERENCE TO RELATED APPLICATIONS

This application is a National Stage application of International Application No. PCT/EP2007/008840, filed on Oct. 11, 2007, which claims priority of German application number 10 2006 048 816.4, filed on Oct. 16, 2006, both of which are incorporated herein by reference in their entireties.

### BACKGROUND OF THE INVENTION

1. Field of the Invention

The present invention relates to a device for locally producing microwave plasmas. The device comprises at least one microwave feed that is surrounded by at least one dielectric tube. The present invention further relates to a method for locally producing microwave plasmas by using said device.

2. Description of the Prior Art

Devices for generating microwave plasmas are being used in the plasma treatment of workpieces and gases. Plasma treatment is used, for example, for coating, cleaning, modifying and etching of workpieces, for treating medical implants, for treating textiles, for sterilisation, for light generation, preferably in the infrared to ultraviolet spectral range, for converting gases or for gas synthesis, as well as in waste gas purification technology. To this end, the workpiece or gas to be treated is brought into contact with the plasma or the microwave radiation.

The geometry of the workpieces to be treated ranges from flat substrates, fibres and webs, to any configuration of shaped articles.

The most important process gases are inert gases, fluorine-containing and chlorine-containing gases, hydrocarbons, furans, dioxins, hydrogen sulfides, oxygen, hydrogen, nitrogen, tetrafluoromethane, sulfur hexafluoride, air, water, and mixtures thereof In the purification of waste gases by means of microwave-induced plasma, the process gas consists of all kinds of waste gases, especially carbon monoxide, hydrocarbons, nitrogen oxides, aldehydes and sulfur oxides. However, these gases can be used as process gases for other applications as well.

Devices that generate microwave plasmas have been described in the documents WO 98/59359 A1, DE 198 480 22 A1 and DE 195 032 05 C1.

The above-listed documents have in common that they describe a microwave antenna in the interior of a dielectric tube. If microwaves are generated in the interior of such a tube, surface waves will form along the external side of that tube. In a process gas which is under low pressure, these surface waves produce a linear elongate plasma. Typical low pressures are 0.1 mbar-10 mbar. The volume in the interior of the dielectric tube is typically under ambient pressure (generally normal pressure; approximately 1013 mbar). In some embodiments a cooling gas flow passing through the tube is used to cool the dielectric tube.

To feed the microwaves, hollow waveguides and coaxial conductors are used, inter alia, while antennas and slots, among others, are used as the coupling points in the wall of the plasma chamber. Such feed lines for microwaves and coupling points are described, for example, in DE 423 59 14 and WO 98/59359 A1.

The microwave frequencies employed for generating the plasma are preferably in the range from 800 MHz to 2.5 GHz, more preferably in the ranges from 800 MHz to 950 MHz and 2.0-2.5 GHz, but the microwave frequency may lie in the entire range from 10 MHz up to several 100 GHz.

DE 198 480 22 A1 and DE 195 032 05 C1 describe devices for the production of plasma in a vacuum chamber by means of electromagnetic alternating fields, comprising a conductor that extends, within a tube of insulating material, into the vacuum chamber, with the insulating tube being held at both ends by walls of the vacuum chamber and being sealed with respect to the walls at its outer surface. The ends of the conductor are connected to a generator for generating the electromagnetic alternating fields.

A device for producing homogenous microwave plasmas according to WO 98/59359 A1 enables the generation of particularly homogeneous plasmas of great length, even at higher process pressures, as a result of the homogeneous input coupling of the microwaves.

The possible applications of the above-mentioned plasma sources are limited by the high energy release of the plasma to the dielectric tube. This energy release may result in an excessive heating of the tube and ultimately lead to the destruction thereof For that reason, these sources are typically operated at microwave powers of about 1-2 kW at a correspondingly low pressure (approximately 0.1-0.5 mbar). The process pressures can also be 1 mbar-100 mbar, but only under certain conditions and at a correspondingly low power, in order not to destroy the tube.

With the above-mentioned devices, typical plasma lengths of 0.5-1.5 m can be achieved. With plasmas of almost 100% argon it is possible to achieve greater lengths, but such plasmas are of little technical importance.

Another problem with such plasma sources lies in the radially symmetrical radiation of microwaves and the associated radially symmetrically radiated power in applications where only a delimited angular region of the plasma source is needed. Any power that is radiated into another angular region than that of the application is lost to the application.

### SUMMARY OF THE PRESENT INVENTION

It is the object of the present invention to overcome the above-mentioned disadvantages and thereby to minimize the portion of the loss power.

In accordance with the invention, this object is achieved by a device for locally generating microwave plasmas. This device comprises at least one microwave feed which is surrounded by at least one dielectric tube. At least one of the dielectric tubes, preferably the outer dielectric tube, is partially surrounded by a metal jacket.

By means of the microwave-shielding effect of the metal jacket, the device advantageously enables the generation of a plasma in a region intended therefore and thus prevents the generation of plasma, and thereby power radiation, outside that region.

Suitable microwave feeds are known to those skilled in the art. Generally, a microwave feed consists of a structure which is able to emit microwaves into the environment. Structures that emit microwaves are known to those skilled in the art and can be realised by means of all known microwave antennae and resonators comprising coupling points for coupling the microwave radiation into a space. For the above-described device, cavity resonators, bar antennas, slot antennas, helix antennas and omnidirectional antennas are preferred. Coaxial resonators are especially preferred.

In service, the microwave feed is connected via microwave feed lines (hollow waveguides or coaxial conductors) to a microwave generator (e.g. klystron or magnetron). To control the properties of the microwaves and to protect the elements, it is furthermore possible to introduce circulators, insulators, tuning elements (e.g. 3-pin tuners or E/H tuners) as well as mode converters (e.g. rectangular and coaxial conductors) in the microwave supply.

The dielectric tubes are preferably elongate. This means that the tube diameter : tube length ratio is between 1:1 and 1:1000, and preferably 1:10 to 1:100. Furthermore, the tubes are preferably straight, but they may also be of a curved shape or have angles along their longitudinal axis.

The cross-sectional surface of the tubes is preferably circular, but generally any desired surface shapes are possible. Examples of other surface shapes are ellipses and polygons.

The elongate shape of the tubes produces an elongate plasma. An advantage of elongate plasmas is that by moving the plasma device relative to a flat workpiece it is possible to treat large surfaces within a short time.

The dielectric tubes should, at the given microwave frequency, have a low dielectric loss factor tan δ for the microwave wavelength used. Low dielectric loss factors tan δ are in the range from 10⁻² to 10⁻⁷.

Suitable dielectric materials for the dielectric tubes are metal oxides, semimetal oxides, ceramics, plastics, and composite materials of these substances. Particularly preferred are dielectric tubes made of silica glass or aluminium oxide with dielectric loss factors tan δ in the range from 10⁻³ to 10⁻⁴. The dielectric tubes here may be made of the same material or of different materials.

The metal jacket surrounds at least one dielectric tube and partially covers same. The metal jacket has the effect of a microwave shield and prevents the radiation of microwaves into the angular region that is covered by the metal jacket.

The metal jacket preferably consists of a metal of good electric conductivity and with a specific resistance that is smaller than 50 Ω·mm²/m, preferably smaller than 0.5 Ω·mm²/m. Particularly preferred is a metal that, in addition to good electric conductivity characteristics, has good thermal conductivity characteristics, with a thermal conductivity coefficient greater than 10 W/(m·K), more preferably greater than 100 W/(m·K). For economic reasons, the ultimate limit for the above-mentioned values may be 0 Ω·mm²/m for the specific resistance (superconductor) and 10000 W/(m·K) for the thermal conductivity coefficient. Such a metal may be a pure metal or an alloy and may contain, for example, silver, copper, iron, aluminium, chromium or vanadium.

The shape of the metallic jacket is preferably conformed to the outer contour of the dielectric tube, and may be made, for example, of a metallic tube, a bent sheet metal, a metal foil, or a metallic layer, and may be plugged or electroplated thereon, or applied thereon in another way.

The metal jacket region of the dielectric tube that is not shielded, in the following also referred to as “free region”, may be of any shape. Preferably, the free region extends over the entire length of the tube and, in a particularly preferred embodiment, is rectilinearly delimited. The invention comprises further embodiments with all kinds of shapes of apertures, e.g. holes, slots, regular, irregular and curved edge delimitations.

Such metallic microwave shields are capable of limiting the angular region in which the plasma generation takes place in any way desired and thereby reduce the power requirement correspondingly. The angle of aperture within which the microwaves leave the shield may take any value smaller than 360°. Angles of aperture of less than 180° are preferred, especially preferably less than 90°.

By means of the metal jacket it is possible to treat broad webs of material with plasma at a low power loss. The metal jacket shields that spatial region of the device which does not face the workpiece, and there is generated only a narrow plasma strip between the workpiece and the device, preferably over the entire width of the workpiece.

The plasma treatment of a workpiece can also, in addition to a static plasma treatment, be carried out by moving the device relative to a workpiece or a surface. This movement may be parallel to the longitudinal direction of the dielectric tube, but is preferably non-parallel to the longitudinal direction of the dielectric tube, more preferably orthogonal to said longitudinal direction.

According to one particular embodiment, the dielectric tubes are closed at their end faces by walls.

A gas-tight or vacuum-tight connection between the tubes and the walls is advantageous. Connections between two workpieces are known to those skilled in the art and may, for example, be glued, welded, clamped or screwed connections. The tightness of the connection may range from gas-tight to vacuum-tight, with vacuum-tight meaning, depending on the working environment, tightness in a rough vacuum (300-1 hPa), fine vacuum (1-10⁻³ hPa), high vacuum (10⁻³-101⁻⁷ hPa) or ultrahigh vacuum (10⁻⁷-10⁻¹² hPa). Generally, the term “vacuum-tight” here refers to tightness in a rough or fine vacuum.

The walls may be provided with passages, through which a dielectric fluid can be conducted in order to cool the dielectric tube. Both a gas and a dielectric liquid may be used as the dielectric fluid.

To keep the heating of the fluid by the microwaves as low as possible, the fluid must, at the wavelength of the microwaves, have a low dielectric loss factor tan δ in the range of from 10⁻² to 10⁻⁷. This prevents a microwave power input into the fluid or reduces said input to an acceptable degree.

An example of a dielectric liquid is an insulating oil such as, for instance, mineral oils, olefins (e.g. poly-alpha-olefin) or silicone oils (e.g. COOLANOL® or dimethyl polysiloxane).

By means of this fluid cooling of the outer dielectric tube, it is possible to reduce the heating of the outer dielectric tube. This enables higher microwave powers which, in turn, lead to an increase in the concentration of the plasma at the outside of the outer dielectric tube. In addition, the cooling enables a higher process pressure than in uncooled plasma generators.

In a preferred embodiment according to the invention, the material of the outer dielectric tube is replaced by a porous dielectric material. Suitable porous dielectric materials are ceramics or sintered dielectrics, preferably aluminium oxide. However, it is also possible to provide tube walls of silica glass or metal oxides with small holes.

When a gas flows through the dielectric tubes, part of the gas escapes through said pores. Since the highest microwave field strengths are present at the surface of the outer dielectric tube, the gas molecules, upon passing through the outer dielectric tube, travel through the zone of the highest ion density.

Furthermore, after passing through the pores, the gas has a resultant movement direction radially away from the tube.

If the same gas is used for cooling as is used as the process gas, the portion of the excited particles is increased by the passage of the process gas through the region of the highest microwave intensity. In this way, an efficient transport of excited particles to the workpiece is ensured. This increases both the concentration and the flow of the excited particles.

Any known gas may be used as the process gas. The most important process gases are inert gases, fluorine-containing and chlorine-containing gases, hydrocarbons, furans, dioxins, hydrogen sulfides, oxygen, hydrogen, nitrogen, tetrafluoromethane, sulfur hexafluoride, air, water, and mixtures thereof. In the purification of waste gases by means of microwave-induced plasmas, the process gas consists of all kinds of waste gases, especially carbon monoxide, hydrocarbons, nitrogen oxides, aldehydes and sulfur oxides. However, these gases can be used as process gases for other applications as well.

All of the above-described devices for plasma generation, during operation, form a plasma at the outer side of the dielectric tube which is not shielded by the metal jacket.

In a normal case, the device will be operated in the interior of a space (plasma chamber). This plasma chamber may have various shapes and apertures and serve various functions, depending on the operating mode. For example, the plasma chamber may contain the workpiece to be processed and the process gas (direct plasma process), or process gases and openings for plasma discharge (remote plasma process, waste gas purification).

### BRIEF DESCRIPTION OF THE DRAWINGS

In the following, the invention will be explained, by way of example, by means of the embodiments which are schematically represented in the drawings.

FIG. 1A is a cross-section sectional view of the device according to the present invention.

FIG. 1B is perspective view of the device according to the present invention.

FIGS. 2A to 2D show, in lateral view, various examples of shapes of the above-described device.

FIG. 3A is a perspective view of a possible embodiment of the present invention for treating large-area workpieces.

FIG. 3B cross-sectional view of the embodiment of the present invention for treating large-area workpieces as shown in FIG. 3A.

### DETAILED DESCRIPTION OF THE PRESENT INVENTION

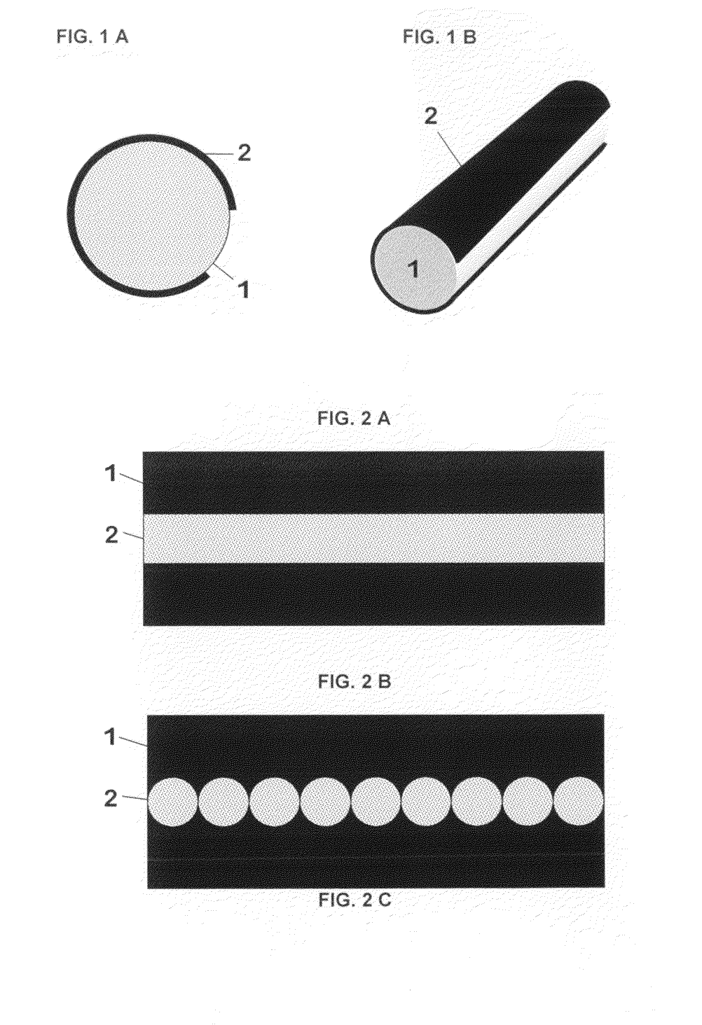

FIGS. 1A and 1B show a cross-section and a perspective view of a device for locally generating microwave plasmas, wherein a dielectric tube (1), which contains the microwave feed and optionally further elements and tubes (not shown), is surrounded by a metal jacket (2), such that a region of approximately 320° is shielded by the metal jacket. The dielectric tube may, in addition to the microwave feed, contain further elements, such as cooling medium or further tubes.

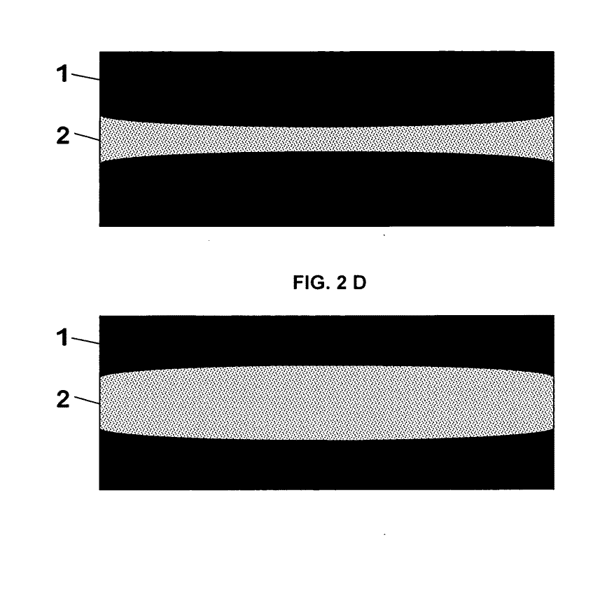

FIGS. 2A to 2D show, in side view, various examples of the shape of the region of the dielectric tube (1) that is not covered by the metal jacket (2). These drawings are to be understood as developed lateral surfaces of a cylindrical dielectric tube and the metal jacket.

FIG. 2A shows a rectangular region,

FIG. 2B shows a region consisting of round surfaces,

FIG. 2C shows a biconcave surface, and

FIG. 2D shows a biconvex surface.

In addition to these examples, any conceivable shape of the non-covered area are possible.

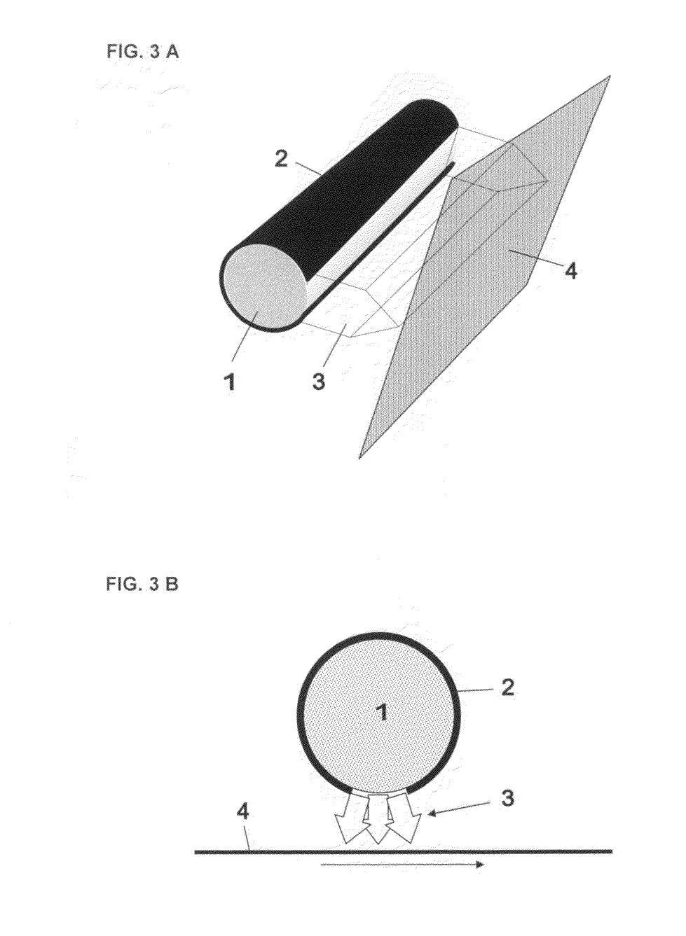

FIGS. 3A and 3B show, in a perspective representation and in a cross-section, a device for the local generation of microwave plasmas, wherein the major part of the lateral surface of the outer dielectric tube (1) is enclosed by a metal jacket (2), and a plasma (3), depicted in the drawing by transparent arrows, that can only be formed in a narrow region. In this region, a workpiece (4), moving relative to the device, can be treated with the plasma over a large surface area.

All of the embodiments are fed by a microwave supply, not shown in the drawings, consisting of a microwave generator and, optionally, additional elements. These elements may comprise, for example, circulators, insulators, tuning elements (e.g. three-pin tuner or E/H tuner) as well as mode converters (e.g. rectangular or coaxial conductors).

There are numerous fields of application for the above described device and the above described method. Plasma treatment is employed, for example, for coating, cleaning, modifying and etching of workpieces, for the treatment of medical implants, for the treatment of textiles, for sterilisation, for light generation, preferably in the infrared to ultraviolet spectral region, for conversion of gases or for the synthesis of gases, as well as in gas purification technology. The workpiece or gas to be treated is brought into contact with the plasma or microwave radiation. The geometry of the workpieces to be treated ranges from flat substrates, fibres and webs to shaped articles of any shape.

Due to the increased density of the excited particles and to the increased plasma power, it is possible to achieve higher process velocities than with devices and methods according to the prior art.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

I claim:

1. A device for plasma treatment of a workpiece by locally generating microwave plasmas, said device comprising at least one microwave feed and at least one dielectric tube for surrounding said at least one microwave feed, said at least one dielectric tube including an outer dielectric tube, and further comprising a metal jacket for partially surrounding at least one of said at least one dielectric tube, said metal jacket comprising one selected from the group consisting of a metal tube, a bent sheet metal, a metal foil and a metallic layer, said metal jacket leaving free a region of the lateral surface of said at least one dielectric tube that has an angle of aperture of less than 360°, said free region facing the workpiece.

2. The device according to claim 1, wherein the metal jacket comprises a metal with good electric conductivity having a specific resistance that is smaller than 50 Ω·mm²/m.

3. The device according to claim 1, wherein the metal jacket comprises a metal having good thermal conductivity characteristics with a thermal conductivity coefficient greater than 10 W/(m·K).

4. The device according to claim 1, wherein said metal jacket comprises pure metal or an alloy.

5. The device according to claim 1, wherein said metal jacket is conformed to the outer contour of the at least one dielectric tube.

6. The device according to claim 1, wherein said metal jacket is plugged or electroplated onto said device.

7. The device according to claim 1, wherein said metal jacket leaves free a region of the lateral surface of the at least one dielectric tube, said region extending over the entire length of the at least one dielectric tube or comprises holes or slits.

8. The device according to claim 1, wherein said metal jacket does not cover a region with an angle of aperture of less than 180° for facilitating the exit of the microwaves.

9. The device according to claim 1, wherein at least one of the dielectric tubes is made of materials selected from the group consisting of metal oxides, semimetal oxides, ceramics, plastics, and composite materials of these substances.

10. The device according to claim 1, wherein at least one of said at least one dielectric tube is cooled by a fluid.

11. The device according to claim 1, wherein the outer dielectric tube is porous or gas-permeable at least in a partial region of the lateral surface or in the region of the entire lateral surface.

12. The device according to claim 1, further comprising a process chamber outside the metal jacket.

13. The device according to claim 1, wherein said at least one microwave feed is selected from the group consisting of a microwave antenna and a cavity resonator with coupling points.

14. The device according to claim 1, further comprising microwave feed lines and a microwave generator, said microwave feed lines connecting said at least one microwave feed, with said microwave generator.

15. A method for locally generating microwave plasmas in a device for the plasma treatment of a workpiece, said device comprising at least one microwave feed that is surrounded by at least one dielectric tube, wherein part of the microwave power is shielded by a metal jacket that comprises one selected from the group consisting of a metallic tube, a bent sheet metal, a metal foil and a metallic layer, said metal jacket partially enclosing at least one of said at least one dielectric tube, and wherein said metal jacket leaves free a region of the lateral surface of said at least one dielectric tube that has an angle of aperture of less than 360°, said free region facing the workpiece.

16. The method according to claim 15, wherein a spatial region of the device does not face the workpiece, said method comprising the step of shielding said spatial region of the device which does not face the workpiece against the exist of the microwaves with said metal jacket.

17. The method according to claim 15, comprising the step of forming the plasma in a region with an angle of aperture of less than 180° wherein said region is not covered by the metal jacket.

18. The method according to claim 15, further comprising the step of moving a workpiece or a surface relative to the at least one dielectric tube, said movement being parallel to or not parallel to the longitudinal direction of the at least one dielectric tube.

19. The method according to claim 18, wherein the movement is not parallel to the longitudinal direction of the at least one dielectric tube, with the direction of movement being orthogonal to the longitudinal direction of the at least one dielectric tube.

20. The method according to claim 15, further comprising the step of cooling at least one of the at least one dielectric tube by a fluid that has a low dielectric loss factor tan δ in the range of from 10⁻² to 10⁻⁷.

21. Use of a device comprising at least one microwave feed and at least one dielectric tube for surrounding said at least one microwave feed, said at least one dielectric tube including an outer dielectric tube, and further comprising a metal jacket for partially surrounding at least one of said at least one dielectric tube, said metal jacket comprising one selected from the group consisting of a metal tube, a bent sheet metal, a metal foil and a metallic layer, said metal jacket leaving free a region of the lateral surface of said at least one dielectric tube that has an angle of aperture of less than 360°, said free region facing the workpiece, said device for generating a plasma for coating, cleaning, modifying and etching of workpieces, for treating medical implants, for treating textiles, for sterilisation, for light generation, for light generation in the infrared to ultraviolet spectral region, for converting gases or for gas synthesis, as well as in waste gas purification technology.

22. Use of a device according to claim 15 for generating a plasma for coating, cleaning, modifying and etching of workpieces, for treating medical implants, for treating textiles, for sterilisation, for light generation, for light generation in the infrared to ultraviolet spectral region, for converting gases or for gas synthesis, as well as in waste gas purification technology.

23. The device according to claim 1, wherein said metal jacket partially surrounds said outer dielectric tube.

24. The device according to claim 2, wherein the metal jacket comprises a metal with good electric conductivity having a specific resistance that is smaller than 0.5 Ω·mm²/m.

25. The device according to claim 3, wherein the metal jacket comprises a metal having good thermal conductivity characteristics with a thermal conductivity coefficient greater than 100 W/(m·K.).

26. The device according to claim 4, wherein said metal jacket comprises a metal selected from the group consisting of silver, copper, iron, aluminium, chromium and vanadium.

27. The device according to claim 7, wherein said region comprises delimitations having a configuration selected from the group consisting of rectilinear, regular, irregular and curved edge delimitations.

28. The device according to claim 8, wherein said metal jacket does not cover a region with an angle of aperture of less than 90° for facilitating the exit of the microwaves.

29. The device according to claim 9, wherein at least one of the dielectric tubes is made of materials selected from the group consisting of silica glass and aluminium oxide.

30. The device according to claim 13, wherein said at least one microwave feed is a coaxial resonator.

31. The device according to claim 14, wherein said microwave feed lines are selected from the group consisting of hollow waveguides and coaxial conductors, and wherein said microwave generator is selected from the group consisting of a klystron and a magnetron.

32. The method according to claim 17, comprising the step of forming the plasma in a region with an angle of aperture of less than 90°, wherein said region is not covered by the metal jacket.
